(19) United States
(12) Patent Application Publication
(10) Pub. No.: US 2026/0363619 A1
(43) Pub. Date: Nov. 19, 2026

(54) INTRAORAL MULTISENSOR DEVICE AND SYSTEM

(71) Applicants: Matthew Cathlina, Libertyville, IL (US); Patrick Kennedy, Libertyville, IL (US)

(72) Inventors: Matthew Cathlina, Libertyville, IL (US); Patrick Kennedy, Libertyville, IL (US)

(21) Appl. No.: 19/207,186

(22) Filed: May 13, 2025

(51) Int. Cl. A61B 5/00; A61B 5/145; A61B 7/00; A61B 90/00; A61C 7/08; A63B 71/08

(52) U.S. Cl. CPC A61B 5/682; A61B 5/14539; A61B 5/4557; A61B 5/4818; A61B 5/7282; A61B 5/7455; A61B 5/747; A61B 7/003; A61B 2090/064; A61C 7/08; A63B 71/085

(57) ABSTRACT

A non-invasive intraoral multisensor device and system for measuring, recording, organizing, and communicating the information and data collected from said device for real-time monitoring and analysis of a user's health metrics and health status obtained from a user's oral cavity. The system comprises an instrumented mouth guard device, an intraoral multisensor circuitry system (IMCS) coupled to the instrumented mouth guard device, and a remote receiving device. The intraoral multisensory system is capable of detecting and measuring collision impact forces, collecting time series of force and pH measurements, and facilitating automated therapeutic interventions, such as vibration modes, for conditions like bruxism and sleep apnea and aims to analyze, diagnose, and monitor a user's health metrics and status, offering a comprehensive solution for oral health management.

### TECHNICAL FIELD

This disclosure relates to wearable orthodontic smart devices and systems for sensing health metrics and data of a device user and, more particularly, to an intraoral multisensor device and system adapted to provide non-invasive, real-time health metrics and biological data obtained from a user's oral cavity to analyze, diagnose and monitor a user's health metrics and health status.

### BACKGROUND

Orthodontic devices, in particular retainers and aligners, are primarily and traditionally utilized in orthodontic appliances to exert an applied force onto an individual's teeth to reposition the individual's teeth into a desired arrangement and correct malocclusions and/or improve orthodontic aesthetics. Mouth guards, however, are multifunctional dental appliances applicable and utilized in a wide variety of activities ranging from contact sports to sleep disorder treatment. In addition to its utilization in a variety of categorical activities, mouth guards present a spectrum of available designs and accessibility from general ready-made stock mouth guards, adaptable boil-and-bite mouth guards, and custom-made mouth guards prepared from an individual's dental impressions to fit their precise dental anatomy. However, these devices and/or systems fail to provide an intraoral multisensor device, method and system capable of data collection and automated, customizable therapeutic activation for various dental, sport, and health monitoring applications. Accordingly, there is a need in the pertinent art for devices, systems, and methods for measuring and monitoring real-time health metrics and biological data obtained in a non-intrusive manner from a user's oral cavity to analyze, diagnose, and monitor said user's health metrics and health status.

### SUMMARY OF THE INVENTION

This disclosure relates generally to a non-invasive intraoral multisensor device and a system. The teachings herein can provide measuring, recording, organizing and communicating information and data collected from said device for real-time monitoring and analysis of a user's health status.

One implementation of a method for processing, storing, and transmitting data associated with a user includes fabricating an instrumented mouth guard device based on at least one of intraoral dimensions of the user and a specific need of the user; identifying at least one component engagement site on the instrumented mouth guard device based on the specific need of the user; mounting at least one intraoral multisensor circuitry system (IMCS) to the at least one component engagement site of the instrumented mouth guard device based on health monitoring needs of the user; and monitoring the data received from the instrumented mouth guard device for an event involving the user, the instrumented mouth guard device adapted to initiate at least one mechanical feedback indicator on a remote receiving device in response to the event.

One implementation of a non-invasive intraoral multisensor system for processing, storing, and transmitting biological data associated with a user includes an instrumented mouth guard device adapted to at least one of receive, process, store, and transmit the biological data, monitor at least one physiological parameter of the user; and a remote receiving device in communication with the instrumented mouth guard device, the remote receiving device adapted to at least one of receive, process, and store the biological data received from the instrumented mouth guard device, and initiate therapeutic intervention using the instrumented mouth guard device.

Variations in these and other aspects of the disclosure will be described in additional detail hereafter.

### BRIEF DESCRIPTION OF THE DRAWINGS

The various features, advantages, and other uses of the device, system, and methods will become more apparent by referring to the following detailed description and drawings, wherein like reference numerals refer to like parts throughout the several views. It is emphasized that, according to common practice, the various features of the drawings are not to-scale. On the contrary, the dimensions of the various features are arbitrarily expanded or reduced for clarity.

FIG. 1 is a schematic illustration of a first illustrated embodiment of an intraoral multisensor device and system, showing an instrumented mouth guard device, a remote receiving device, and a communication channel, in accordance with implementations of this disclosure;

FIG. 2A is a three-dimensional top elevation view of a first illustrated embodiment of an instrumented mouth guard device worn by a user, showing a main aligner body, a channel, and two component engagement sites, in accordance with implementations of this disclosure;

FIG. 2B is a three-dimensional top elevation view of the first illustrated embodiment of the instrumented mouth guard device worn by the user, showing the main aligner body, the channel, and three component engagement sites, in accordance with implementations of this disclosure;

FIG. 2C is a three-dimensional top elevation view of the first illustrated embodiment of the instrumented mouth guard device worn by the user, showing the main aligner body, the channel, and four component engagement sites, in accordance with implementations of this disclosure;

FIG. 2D is a three-dimensional top elevation view of the first illustrated embodiment of the instrumented mouth guard device worn by the user, showing the main aligner body, the channel, and four component engagement sites, in accordance with implementations of this disclosure;

FIG. 2E is a three-dimensional top elevation view of the first illustrated embodiment of the instrumented mouth guard device worn by the user, showing the main aligner body, the channel, and three component engagement sites, in accordance with implementations of this disclosure;

FIG. 3A is a three-dimensional top elevation view of the first illustrated embodiment of the instrumented mouth guard device worn by the user, showing five component engagement sites, in accordance with implementations of this disclosure;

FIG. 3B is a three-dimensional right side elevation view of the first illustrated embodiment of the instrumented mouth guard device worn by the user in accordance with implementations of this disclosure;

FIG. 3C is a three-dimensional front elevation view of the first illustrated embodiment of the instrumented mouth guard device worn by the user in accordance with implementations of this disclosure;

FIG. 3D is a three-dimensional left side elevation view of the first illustrated embodiment of the instrumented mouth guard device worn by the user in accordance with implementations of this disclosure;

FIG. 3E is a three-dimensional top elevation view of the first illustrated embodiment of the instrumented mouth guard device worn by the user, showing two component engagement sites, in accordance with implementations of this disclosure;

FIG. 3F is a three-dimensional right side elevation view of the first illustrated embodiment of the instrumented mouth guard device worn by the user in accordance with implementations of this disclosure;

FIG. 3G is a three-dimensional front elevation view of the first illustrated embodiment of the instrumented mouth guard device worn by the user in accordance with implementations of this disclosure;

FIG. 3H is a three-dimensional left side elevation view of the first illustrated embodiment of the instrumented mouth guard device worn by the user in accordance with implementations of this disclosure;

FIG. 3I is a three-dimensional top elevation view of the first illustrated embodiment of the instrumented mouth guard device worn by the user, showing three component engagement sites, in accordance with implementations of this disclosure;

FIG. 3J is a three-dimensional right side elevation view of the first illustrated embodiment of the instrumented mouth guard device worn by the user in accordance with implementations of this disclosure;

FIG. 3K is a three-dimensional front elevation view of the first illustrated embodiment of the instrumented mouth guard device worn by the user in accordance with implementations of this disclosure;

FIG. 3L is a three-dimensional left side elevation view of the first illustrated embodiment of the instrumented mouth guard device worn by the user in accordance with implementations of this disclosure;

FIG. 3M is a three-dimensional top elevation view of the first illustrated embodiment of the instrumented mouth guard device worn by the user, showing four component engagement sites, in accordance with implementations of this disclosure;

FIG. 3N is a three-dimensional right side elevation view of the first illustrated embodiment of the instrumented mouth guard device worn by the user in accordance with implementations of this disclosure;

FIG. 3O is a three-dimensional front elevation view of the first illustrated embodiment of the instrumented mouth guard device worn by the user in accordance with implementations of this disclosure;

FIG. 3P is a three-dimensional left side elevation view of the first illustrated embodiment of the instrumented mouth guard device worn by the user in accordance with implementations of this disclosure;

FIG. 3Q is a three-dimensional top elevation view of the first illustrated embodiment of the instrumented mouth guard device worn by the user, showing three component engagement sites, in accordance with implementations of this disclosure;

FIG. 3R is a three-dimensional right side elevation view of the first illustrated embodiment of the instrumented mouth guard device worn by the user in accordance with implementations of this disclosure;

FIG. 3S is a three-dimensional front elevation view of the first illustrated embodiment of the instrumented mouth guard device worn by the user in accordance with implementations of this disclosure;

FIG. 3T is a three-dimensional left side elevation view of the first illustrated embodiment of the instrumented mouth guard device worn by the user in accordance with implementations of this disclosure;

FIG. 3U is a three-dimensional top elevation view of the first illustrated embodiment of the instrumented mouth guard device worn by the user, showing four component engagement sites, in accordance with implementations of this disclosure;

FIG. 3V is a three-dimensional right side elevation view of the first illustrated embodiment of the instrumented mouth guard device worn by the user in accordance with implementations of this disclosure;

FIG. 3W is a three-dimensional front elevation view of the first illustrated embodiment of the instrumented mouth guard device worn by the user in accordance with implementations of this disclosure;

FIG. 3X is a three-dimensional left side elevation view of the first illustrated embodiment of the instrumented mouth guard device worn by the user in accordance with implementations of this disclosure;

FIG. 4 is a three-dimensional top elevation view of the first illustrated embodiment of the instrumented mouth guard device worn by the user, showing an inductive charging coil and depressed regions, in accordance with implementations of this disclosure;

FIG. 5 is a three-dimensional top elevation view of the first illustrated embodiment of the instrumented mouth guard device worn by the user, showing an anterior portion of the main aligner body, main aligner body sidewalls, and a data port, in accordance with implementations of this disclosure;

FIG. 6 is a bottom elevation view of the first illustrated embodiment of the instrumented mouth guard device, showing a device proximity indicator, in accordance with implementations of this disclosure;

FIG. 7 is a perspective view of the first illustrated embodiment of the instrumented mouth guard device, showing the device proximity indicator, intraoral sensor(s), and component engagement sites, in accordance with implementations of this disclosure;

FIG. 8 is a field implementation of a first illustrated embodiment of intraoral multisensor circuitry system (IMCS) components, showing a capacitive sensor, intraoral sensors, a proximity indicator, a power supply, a microprocessing unit, and a mechanical feedback indicator, in accordance with implementations of this disclosure;

FIG. 9 is a field implementation of an exemplary intraoral multisensor device in communication with an external processing unit in accordance with implementations of this disclosure;

FIG. 10 is a field implementation of an exemplary intraoral multisensory device architecture, showing an impact sensory array, in accordance with implementations of this disclosure;

FIG. 11A is a field implementation of a first illustrated embodiment of a user interface of an application program displayed on a remote electronic device, showing a force map, in accordance with implementations of this disclosure;

FIG. 11B is a field implementation of the first illustrated embodiment of the user interface of the application program displayed on the remote electronic device, showing a force chart, in accordance with implementations of this disclosure;

FIG. 11C is a field implementation of the first illustrated embodiment of the user interface of the application program displayed on the remote electronic device, showing a home screen, in accordance with implementations of this disclosure;

FIG. 11D is a field implementation of the first illustrated embodiment of the user interface of the application program displayed on the remote electronic device, showing a usage screen, in accordance with implementations of this disclosure;

FIG. 12 is a flow diagram of a first illustrated embodiment of a process for making and using an intraoral multisensory device in accordance with implementations of this disclosure;

FIG. 13 is a flow diagram of a process for using and syncing data from the first illustrated embodiment of the instrumented mouth guard device, in accordance with implementations of this disclosure; and

FIG. 14 is a flow diagram of a process for using and syncing data from the first illustrated embodiment of the instrumented mouth guard device, allowing the user to activate vibration therapy, in accordance with implementations of this disclosure.

### DETAILED DESCRIPTION

Orthodontic devices, in particular retainers and aligners, are primarily and traditionally utilized in orthodontic appliances to exert an applied force onto an individual's teeth and/or jaw to reposition the individual's teeth into a desired arrangement, correct malocclusions, expand the jaw, and/or improve orthodontic aesthetics. Mouth guards, however, are multifunctional dental appliances applicable and utilized in a wide variety of activities ranging from contact sports to sleep disorder treatment. In addition to its utilization in a variety of categorical activities, mouth guards present a spectrum of available designs and accessibility from general ready-made stock mouth guards, adaptable boil-and-bite mouth guards, and custom-made mouth guards prepared from an individual's dental impressions to fit their precise dental anatomy, and the like.

Mouth guards, in relation to contact sports, are generally utilized to protect athletes from sports-related injuries and to reduce the risk of chipped or avulsed teeth. More recently, the use of mouth guards has taken on a more important role in contact sports with the technological advancement of smart mouth guards outfitted with sensors that detect and measure the severity of collisions and head impacts. The smart mouthguard sends an alert to sideline staff of said significant impact event warranting removal of the individual from the event to conduct a head injury assessment to clear the athlete's continued participation in the sporting event if a collision impact reaches a designated threshold force. Such smart mouth guards contain embedded inertial measurement units consisting of a linear accelerometer and a gyroscope to calculate force, location, direction, and number of impact events for accurate quantification of head kinematics for identification of underlying brain motion during injurious impacts.

In relation to sleep disorders, mouth guards have been discussed and utilized for diagnosing and managing medical conditions such as Bruxism and Obstructive Sleep Apnea (OSA). Bruxism is a common medical condition characterized by excessive teeth grinding or jaw clenching with significant impact to an individual's dental and physiological health. Night guards, or mouth guards, are widely utilized by individuals with Bruxism as these appliances provide a protective barrier between the upper and lower teeth.

OSA is a medical condition characterized by the complete or partial obstruction of an individual's airway causing repeated lapses in breathing during sleep causing significant health complications most often affecting the circulatory and nervous systems. Treatment for OSA differs among individuals based upon the severity of the condition and may include breathing devices such as a continuous positive airway pressure (CPAP) machine, medication, oral appliances, or even surgery. Oral appliances, or mouthguards, to treat OSA include mandibular advancement devices and tongue-stabilizing devices and work by pulling an individual's jaw or tongue forward to open their airway during sleep. Advancements in mouth guard technology for sleep related disorders focus on diagnostic devices for such disorders and mouth guard systems having measuring and/or monitoring sensors for bite compression forces generated by an individual with Bruxism or respiration levels of an individual with OSA.

Recently, researchers at the Massachusetts Institute of Technology (MIT) Computer Science and Artificial Intelligence Laboratory (CSAIL) and Aarhus University in Denmark created the “MouthIO”, a design and fabrication method for customizing fully self-contained wearable devices that integrate microcontrollers and batteries as the base components and can house a multitude of sensing and actuation components having an open-bite design.

However, these devices and/or systems fail to provide an intraoral multisensor device, method and system capable of data collection and automated, customizable therapeutic activation for various dental, sport, and/or health monitoring applications. Accordingly, there is a need in the pertinent art for devices, systems, and methods for measuring and monitoring real-time health metrics and biological data obtained in a non-intrusive manner from a user's oral cavity to analyze, diagnose, and/or monitor said user's health metrics and health status.

One key aspect of the present disclosure, and widely ignored by the prior art, is the integration and connection of an instrumented and/or smart mouth guard to a computing interface via a wireless and/or wired communication channel. This feature allows data to be collected from various biological sensors, analyzed, and transmitted by a microprocessor to a remote receiving device to monitor, diagnose, and/or implement therapeutic intervention mechanisms within various conditions, activities and/or events. The system of the present disclosure provides mobile application software that allows users to control various features of the intraoral sensing device, review collected data, share said data with family and/or medical professionals and monitor various biological parameters without the need for invasive testing, procedures, and/or a multitude of medical visits.

The system of the present disclosure comprises an instrumented mouth guard device, an intraoral multisensor circuitry system (IMCS) coupled to the instrumented mouth guard device, and a remote receiving device. The IMCS comprises an array of intraoral sensors, a microcontroller unit, a power supply receiver, a mechanical indicator, and an LCD indicator. The intraoral sensors generate sensor signals corresponding to a response generated by the mouth guard wearers activity that exceed a configurable pre-determined threshold. Once a signal is generated, the IMCS may then transmit data describing the wearer's collected health metrics during the duration of the signal to an external remote device for display and analysis.

In one aspect, the system of the present disclosure is directed to an intraoral multisensor device and system capable of detecting and measuring the collision impact force to an athlete's head during contact sports using an array of inertia sensors.

In another aspect, the system of the present disclosure is directed to an intraoral multisensor device and system capable of facilitating collection of time series of force measurements to aid in management and reduction of pain, clenching, grinding, etc.

Yet another aspect of the system of the present disclosure is directed to an intraoral multisensor device and system capable of facilitating collection of time series pH measurements to aid in risk identification of enamel loss and/or pathology.

Yet another aspect of the system of the present disclosure is directed to an intraoral multisensor device and system capable of facilitating automated activation of therapeutic vibration modes when a configurable predetermined force threshold is reached and/or when a configurable predetermined grinding sound threshold is reached. Yet another aspect of the system of the present disclosure is directed to an intraoral multisensor device and system capable of facilitating automated activation of therapeutic vibration modes when a configurable predetermined sound threshold is reached due to snoring to manage sleep apnea.

Yet another aspect of the system of the present disclosure is directed to an intraoral multisensor device and system capable of facilitating salivary testing to aid in health monitoring and/or pathology.

While the present disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

Disclosed herein are various embodiments of the intraoral multisensor device and system for measuring, recording, organizing, and/or communicating information and data collected from said device for real-time monitoring and analysis of a user's health metrics and health status. The intraoral multisensor device and system provides an instrumented mouth guard device integrated and connected to a remote receiving device via a wired and/or wireless communication channel that provides non-invasive, real-time collection of a user's health metrics and biological data obtained from said user's oral cavity and allows for the analysis, diagnosis, and/or monitoring of a user's health metrics and health status. The system's mobile software, executed by a remote receiving and/or electronic device, provides users with full time self-monitoring of their health, identification of impactful health risks, management of health conditions using therapeutic intervention features, and/or gain automatic therapeutic intervention in response to a health status change.

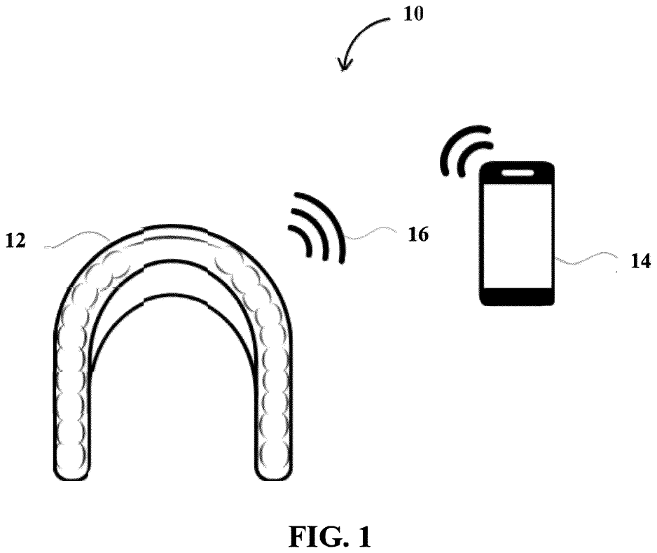

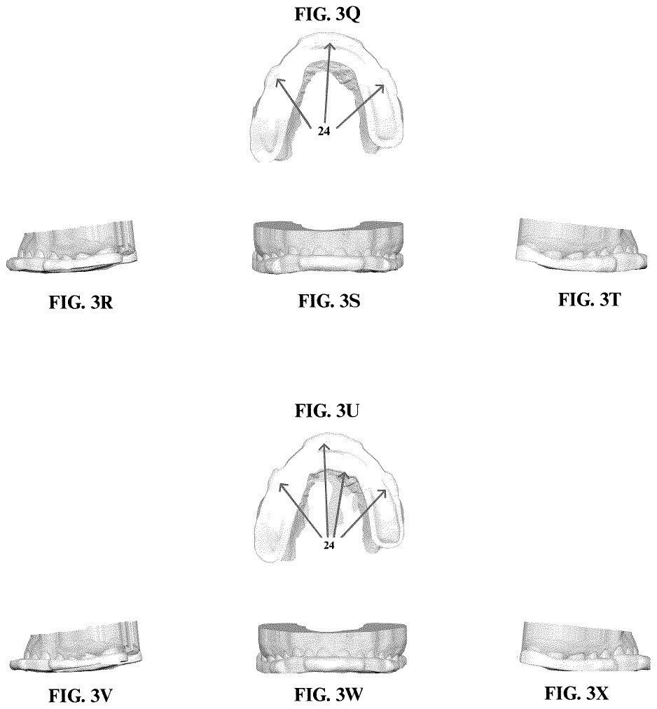

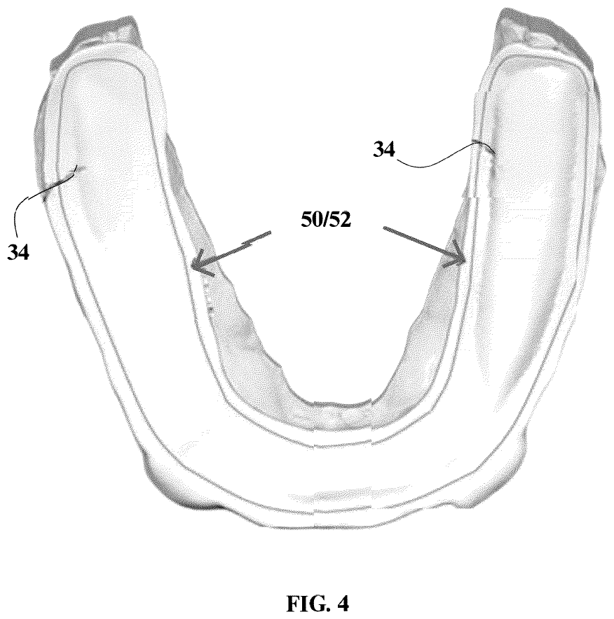

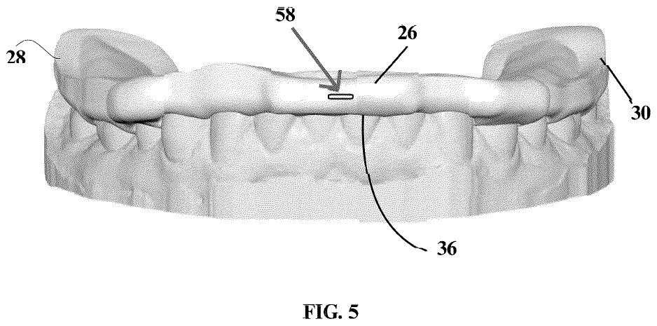

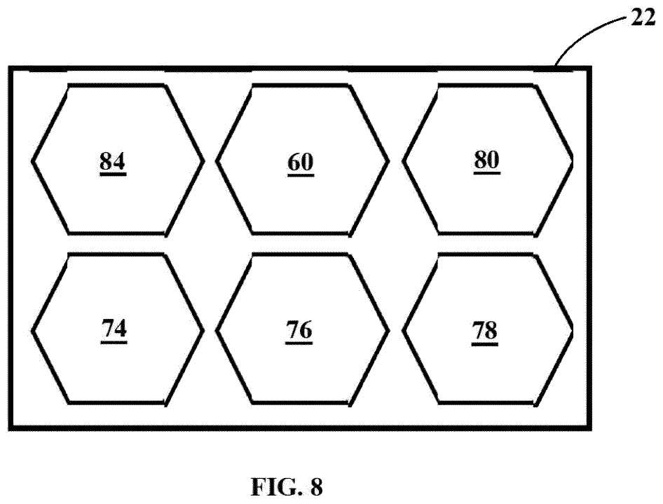

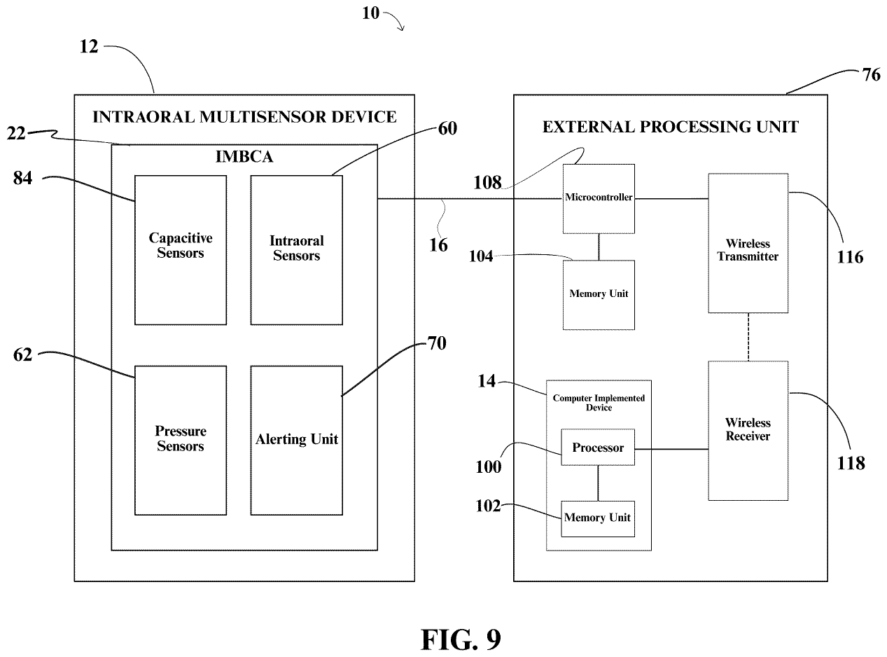

Referring to FIGS. 1-10, a first illustrated embodiment of an intraoral multisensor device and system 10 (FIG. 1) includes an instrumented mouth guard device 12 adapted to detachably engage a portion and/or all of a user's teeth of either the upper, lower, and/or both jaws and a remote receiving and/or electronic device 14, such as a smart phone, tablet, and/or computer, providing non-invasive, real-time health metrics and biological data obtained from the user's oral cavity. The instrumented mouth guard device 12 can connect to the remote receiving device 14 through at least one communication channel 16, such as a wireless communication channel as shown in FIG. 1. The instrumented mouth guard device 12 and the remote receiving and/or electronic device 14 can communicate over a variety of wireless communications links, such as Wi-Fi, cellular, satellite, and/or private wireless systems. The at least one communication channel 16 can be, for example, a wireless local area network (WLAN), wireless metropolitan area network (WMAN), wireless wide area network (WWAN), a private wireless system, a cellular telephone network and/or any other means of transferring data from instrumented mouth guard device 200 to the remote receiving and/or electronic device 14. The communication channel 16 may utilize any communication means suitable for wired and/or wireless transmission of data between the electronic devices, such as a USB cord connected to a USB port 58 (FIG. 5) of the instrumented mouth guard device 12 for wired communication transmission and Bluetooth XX for wireless communication transmission in this exemplary illustrated implementation. When a wireless data connection is not available, the data is stored in local memory until wireless connectivity is restored.

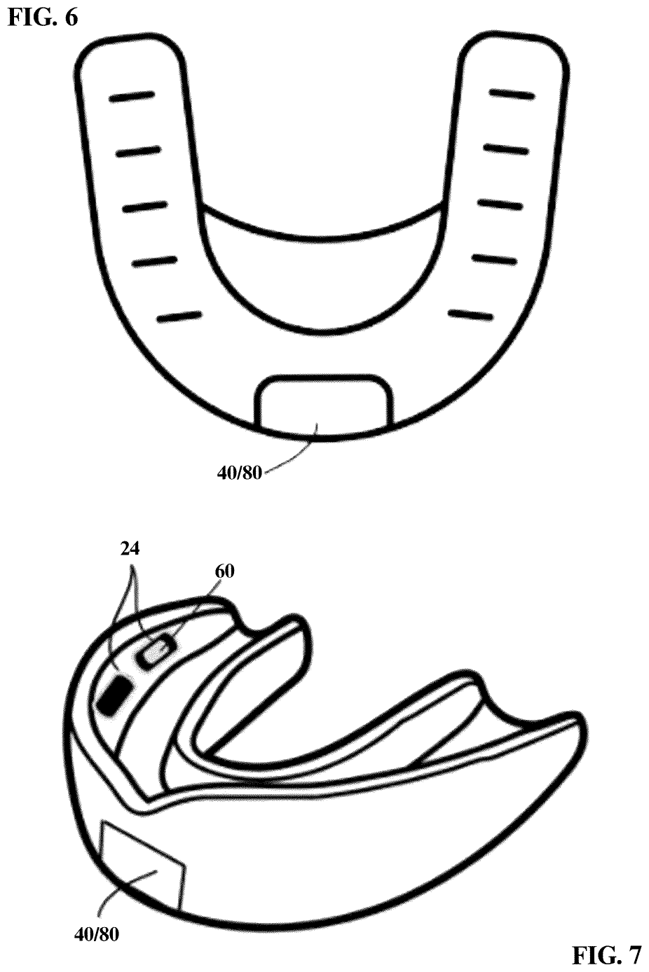

The instrumented mouth guard device 12, as shown in FIGS. 1, 2A-2E, and 3A-3X, includes a main aligner body 20, at least one intraoral multisensor circuitry system (IMCS) component 22 (FIG. 8), and at least one component engagement site(s) 24. In one exemplary illustrated implementation, the instrumented mouth guard device 12 is custom-made and the IMCS 22 components are located and placed on and/or in the instrumented mouth guard device 12 according to the recommendations of a health care or dental provider. The main aligner body 20 is made of a flexible material and comprises an anterior portion 26 with opposing evenly spaced arcuate and/or U-shaped peripheral sidewalls 28, 30 that form a channel 32 and correspond to the contour and dentition of either the upper, lower, or both jaw regions of the user's dental arch. Along the perimeter of the main aligner body 20 and within the channel 32 is a depressed region 34 individually formed using the individual user's dental impression. The depressed region 34 is adapted to receive and engage the user's teeth, thereby aiding in maintaining the instrumented mouth guard device 12 in a fixed position within the user's oral cavity during operational use.

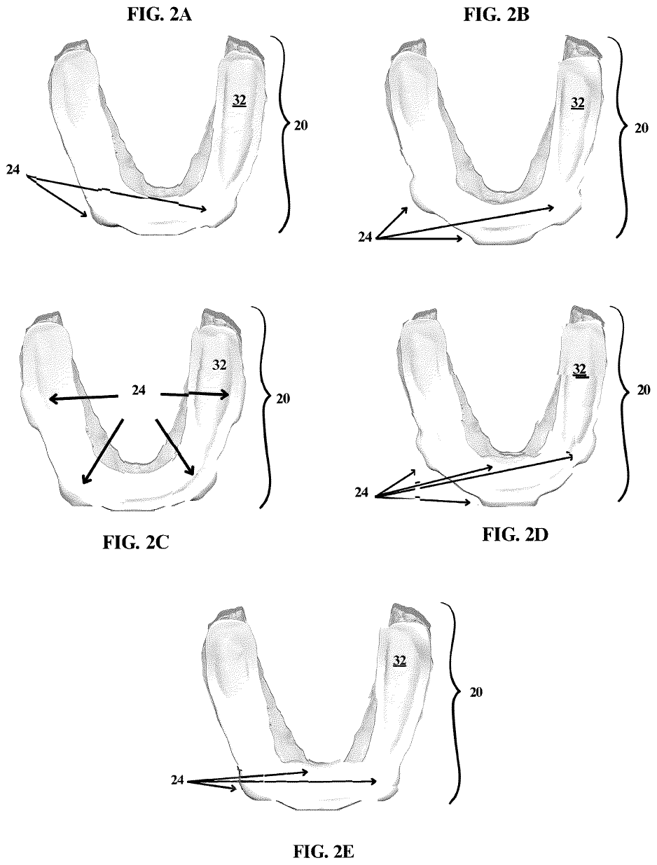

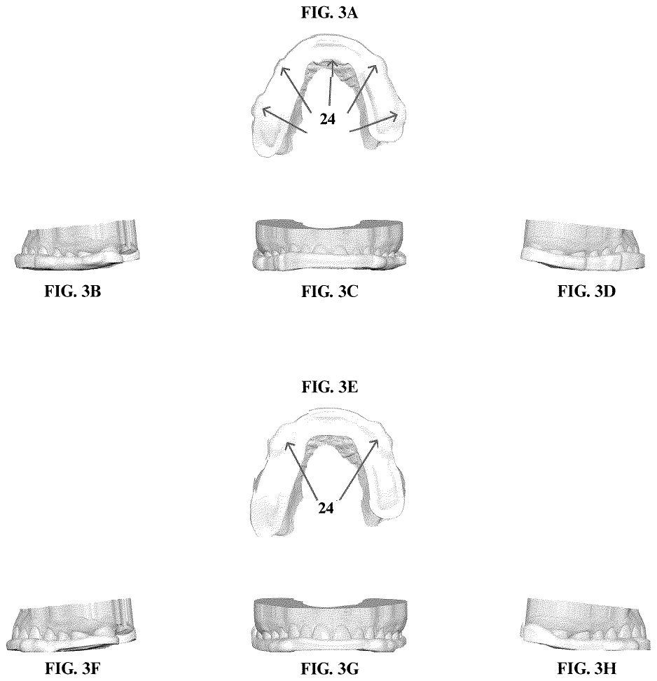

FIGS. 2A-2E and 3A-3X provide three-dimensional (3D) renderings of the instrumented mouth guard device 12 adapted to engage the upper and/or lower jaw of the user. FIGS. 2A-2E provide 3D renderings of the instrumented mouth guard device 12 designed to engage with the lower jaw of a particular user while FIGS. 3A-3X provide 3D renderings of the instrumented mouth guard device 12 designed to engage with the upper jaw of a particular user. Both sets of 3D renderings in FIGS. 2A-2E and FIGS. 3A-3X depict various embodiments of the instrumented mouth guard device 12, comprising modifications in the quantity and placement of the at least one component engagement site(s) 24 adapted to meet the needs of each individual user and house at least one IMCS 22.

The instrumented mouth guard device 12 further includes a control unit 36 disposed at a center-most location of the anterior portion 26 of the main aligner body 20. In certain embodiments of the present disclosure, the control unit 36 includes a power supply component 38 (not shown), a device proximity indicator 40, and a feedback indicator 42 (not shown), such as a mechanical feedback indicator.

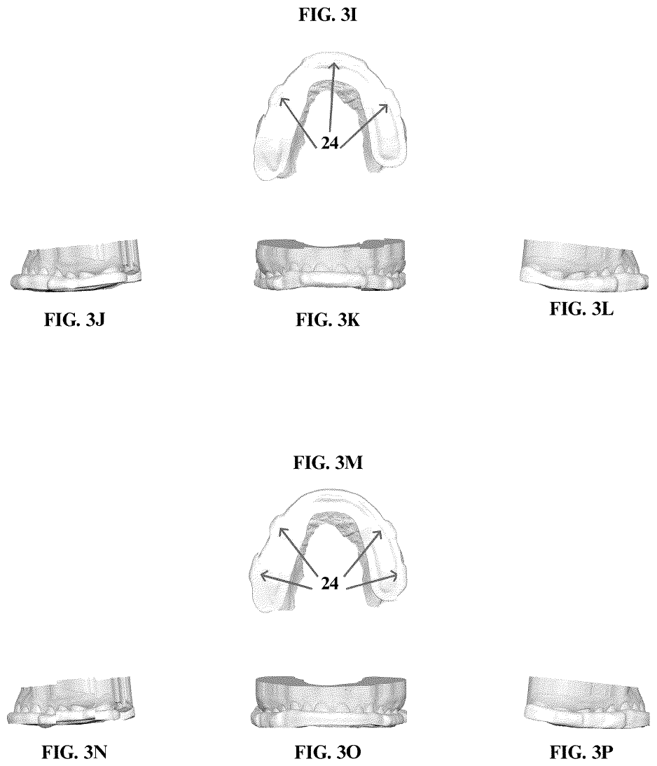

The power supply component 38, in one embodiment of the present disclosure, may comprise a power source 44 (not shown), such as a battery 46 (not shown), and a power receiver 48. The battery 46, in one exemplary illustrated implementation, may be rechargeable using wireless and/or wired means. For example, in the exemplary illustrated implementation depicted in FIG. 4, the power supply component 38 may comprise the power source 44 including the rechargeable battery 46 and the power receiver 48 including a charging circuit 50, such as an inductive coil 52 in this exemplary illustrated implementation, and powers all the electrical components of the IMCS 22.

The instrumented mouth guard device 12 is adapted to include at least one IMCS 22. The at least one IMCS 22 is customized in shape and design such that the IMCS 22 is capable of being placed within channel 32 of the main aligner body 20 while ensuring correspondence to the individual user's dentition and ensuring that the individual user's needs and comfort are met. The at least one IMCS 22 serves as a carrier of various components in a multitude of configurations that are then mounted and housed within the at least one engagement site(s) 24 of the main aligner body 20 for data collection of predetermined health parameters selected based on the user's particular needs. FIG. 8 shows one exemplary illustrated implementation of architectural details and/or components of the IMCS 22 according to particular embodiments of the present disclosure.

Component engagement site(s) 24 are customized and provided at various locations, in various sizes, in various quantities, and in various configurations across the at least one IMCS 22 thereby ensuring that user-specific needs, comfort, and desires are met. For example, and for illustration purposes only, it may be determined that a user may require two, three, or four component engagement site(s) 24 along the main aligner body 20 of the instrumented mouth guard device 12. As depicted by the various embodiments provided by FIGS. 2A-2E and 3A-3X, the component engagement site(s) 24 are adapted to engage with the upper, lower, or both jaw regions of the user to detect and/or monitor various health parameters during a multitude of health-impacting events and/or activities.

The IMCS 22 of the instrumented mouth guard device 12, in one exemplary illustrated implementation, comprises at least one force-sensing unit 54 (not shown), such as a capacitive pressure sensor 56 (not shown). For example, and for illustration purposes only, for users with medical conditions such as Bruxism, the instrumented mouth guard device 12 may comprise four component engagement sites 24 each including IMCS components 22 that include capacitive pressure sensors 56 along the perimeter of the main aligner body 20. The IMCS 22 is capable of collecting, measuring, storing, and transmitting compression and bite force data of the user for a configurable predetermined period of time and analyzing the findings provided by said data. For example and for illustration purposes only, a user seeking to monitor night time bite plate clenching, grinding, and/or bite force and to implement certain intervention therapies may wear the device at night time in their sleep and utilize its features in passive data collection mode where bite force data of the user is collected, transmitted, and stored within the remote receiving or electronic device 14 over a configurable predetermined period of time.

The user may seek to utilize intervention therapies provided in certain embodiments of the present disclosure. For example, the user can utilize a mobile application 18 provided with the system 10 and set various vibration mode settings. In one exemplary illustrated implementation, for example, a health care professional may advise the user that a threshold force of a certain amount may result in permanent damage to the user and must monitor their bite force while asleep. The user can then simply set the bite force threshold in the system's 10 application 18 to a configurable predetermined amount corresponding to their health care professional's recommendations such that if said threshold is reached during the time they are asleep, the mouth guard 12 activates a notification to be sensed by the user and/or to alert the user in the form of the feedback indicator 42, such as the mechanical feedback indicator in this exemplary illustrated implementation. The mechanical feedback indicator 42 may activate vibration forces of a configurable predetermined intensity, duration, and/or mode to relax jaw muscles and remediate the detected event and/or wake the user up and/or otherwise alert the user that an impact event has occurred.

FIG. 8 represents a general schematic illustrating the connectivity of the components mounted on the at least one intraoral multisensor circuit system component (IMCS) 22. The IMCS 22 can include an array and/or variety of additional intraoral sensors 232. The additional intraoral sensors 232, if included, may comprise one or more inertial sensor(s) 62 such as a strain effect or strain gauge force sensor and/or at least one magnetometer 64 coupled to at least one of an accelerometer 66 and a gyroscope 68 adapted to provide data relating to an angular velocity and/or angular acceleration associated with force-impact activities along a symmetrical reference point, such as the center and/or midline,, a pH sensor 70 adapted to collect relative pH levels in the user's saliva to identify pathology and/or detect prospective enamel loss, and/or an auditory sensor 72 (not shown) adapted to provide decibel (dB) sound readings and data detected while the user is grinding their teeth and/or snoring. The IMCS 22 further includes a power supply component 74, a microprocessor unit 76, a feedback indicator 78, such as a mechanical feedback indicator, and a device proximity indicator 80. The IMCS 22 may include sensors to conduct salivary testing to detect oral diseases, microbial populations, hormone levels, cancer risk, and diagnosing and monitoring of other non-oral illnesses.

In one exemplary illustrated implementation, the IMCS 22 components may include at least one force-sensing component 82 (not shown), such as a capacitive sensor 84, adapted to measure the user's grinding, clenching, and/or bite force on bite plates over a configurable predetermined period of time. The capacitive sensor(s) 84 can be used to measure the force the user exerts when biting and/or pressing down on the IMCS 22. Such sensors may be utilized by users with medical conditions such as Bruxism or Obstructive Sleep Apnea for monitoring and control of said condition. The inertial sensors 62 of the IMCS 22 may include the at least one magnetometer 64 coupled to at least one of the accelerometer and the gyroscope adapted to provide data relating to the angular velocity and/or angular acceleration associated with force-impact activities along a symmetrical reference point, such as the center and/or midline, of the instrumented mouth guard device 12. The data provided by the components comprising the inertial sensor(s) 62 may be used to detect and/or provide information relating to the amount, orientation, and/or directional movement data associated with the impact forces experienced by the user during an event.

The microprocessor 76 is operatively connected to and collects data from the at least one intraoral sensor 60, analyzes said data, and transmits said analysis and/or data to the remote receiving or electronic device 14 via the communication channel 16.

In addition to capacitive pressure sensors 84, the IMCS 22 may include an array and/or variety of intraoral sensors 60 to monitor various health parameters of the user. For example, athletes such as football players may design the instrumented mouth guard device 12 to include at least one inertial sensors 60 capable of detecting possible risks of concussions and/or brain injuries when the user experiences a high impact force exceeding a configurable predetermined threshold determined by the user's biometric data or a series of impact forces under a configurable predetermined threshold but of particular concern for a number of other reasons such as the user experiencing a higher amount of force impact within a short period of time. In such instances, the instrumented mouth guard device 12 may be set to provide numerous signal indicators to both the user and those around the user. For example, and for illustration purposes only, the mechanical feedback indicator 42/78 may activate a vibration signal to the user indicating that an impact event occurred and/or may alert the user's coach via the remote receiving or electronic device 14 upon detection of such an event.

Auditory sensors 72 are adapted to measure the intensity of a sound such as snoring or grinding of the user's teeth to diagnose, monitor, and/or alleviate symptoms of medical conditions such as sleep apnea or bruxism. For users with sleep apnea, monitoring the sound intensity of their snoring could provide much insight into any partial or complete obstruction of the user's airway causing repeated lapsed breathing during sleep. Such users can, for example, set the instrumented mouth guard device 12 to activate the mechanical feedback indicator 42/78 upon reaching a configurable predetermined particular sound intensity threshold, thereby sending a vibration signal to the user indicating that an impact event occurred.

The mechanical feedback indicator 42/78 includes a taptic engine 86 (not shown) adapted to implement various vibration modes in response to signal activation for automated therapeutic activation intervention and/or to alert the user or a third party via the instrumented mouth guard device 12 and/or the remote receiving or electronic device 14 that an impact event occurred. The taptic engine 86 includes a plurality of mode settings 88 (not shown) including an operational (on/off) mode 90 (not shown) and twenty various vibration modes 92 (not shown; e.g., different vibration patterns to indicate various events/statuses) that provide vibration intensity ranging from level one to level ten, vibration duration, and the choice to turn the vibration setting on “auto-on” when a particular configurable predetermined threshold is reached.

The proximity indicator 40/80 may comprise an LED driver 94 (not shown) for an LED indicator light 96 (not shown) embedded within the front portion of the instrumented mouth guard device 12 such that, when activated, the LED indicator light 96 is visible to the user and others. The LED indicator light 96 may be used to indicate the user met or exceeded the configurable predetermined threshold such as by signaling that a particular configurable predetermined pH level was detected within the user's oral cavity or may be used to serve as an operational indicator to others and the user (e.g., the device 12 being on/off, the device 12 being properly engaged within the oral cavity, or the battery-charge life of the device 12).

The remote receiving or electronic device 14 comprises a processor 100 including hardware to execute instructions of the software application 18, memory 102 for storing instructions and/or collected data, memory/storage 104 for storing mass data, an input/output (I/O) interface 106 including hardware, software, or both, for communication between one or more I/O interfaces, and a communication interface 122 including hardware, software, or both, for communication between remote receiving or electronic device 14 and at least one other local and/or remote computing devices and/or networks. The remote receiving or electronic device 14 may be, by way of example, a smart phone connected to an application, a tablet, and/or a computer.

The microprocessor unit 76 includes a microcontroller 108 with an embedded analog-to-digital (AD) convertor 112 (not shown) adapted to monitor at least one signal from the at least one intraoral sensors 60 and convert said signals into digital data and a microprocessor 114 (not shown) adapted to then transmit the digital data to the remote receiving or electronic device 14 through a wired telemetry 116 and/or wireless telemetry 118 for data communication between the instrumented mouth guard device 12 and the remote receiving or electronic device 14.

The microprocessor 114, in certain embodiments, may also include a Bluetooth transceiver 120 (not shown) that allows for communication with one or more remote receiving or electronic devices 14 that include software, in the form of an application program, to receive and transmit information with the Bluetooth transceiver 120 within the intraoral multisensor device 12 and system 10 while in use, before use, and/or after use.

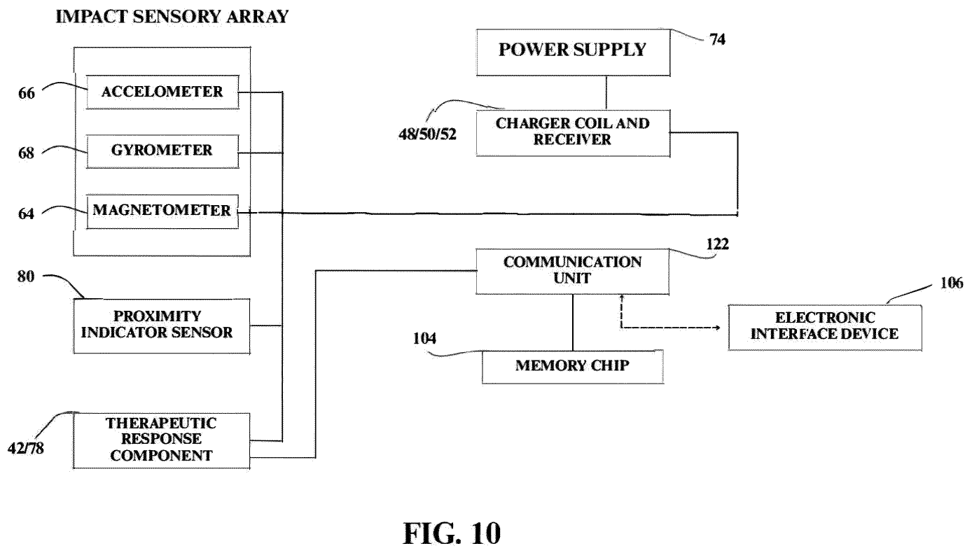

The remote receiving or electronic device 300 is adapted to provide remote storage, computation, and display of risk factors associated with one or more intraoral multisensor devices 12. The remote receiving or electronic device 14 may include a smart phone, computer, tablet, and/or any device capable of receiving and/or transmitting data. One exemplary illustrated implementation of a remote receiving or electronic device 14 is shown in FIGS. 11A-11D.

Other system configurations for the connectivity of the components of the IMCS 22 are possible as well. The IMCS 22 simplifies self-monitoring, provides customized feedback, and intervenes either automatically or by prompting a clinician to intervene in person and/or through the mobile application 18 connected to the system 10.

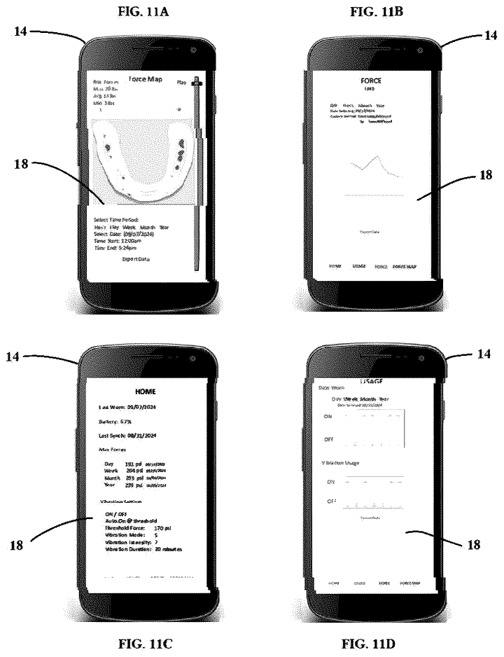

FIG. 12 is a flow diagram showing a first illustrated embodiment of a process 130 for making and using the intraoral multisensory device and system 10 in accordance with an implementation of this disclosure. A user's dental impression is taken to obtain said user's specific intraoral dimensions and an instrumented mouth guard device 12 is fabricated based on said user's dental impression and/or intraoral dimensions 132. At least one component engagement site (IPSS) 24 is coupled to the custom-made instrumented mouth guard device 12 at configurable predetermined locations of the instrumented mouth guard device 12 134. The instrumented mouth guard device 12 collects data associated with the user throughout a configurable predetermined amount of time 136 that the user is wearing the device 12. The instrumented mouth guard device 12 then transmits the data collected to the electronic computing interface 106 for review 138 in real-time and/or after the user is done wearing the device 12.

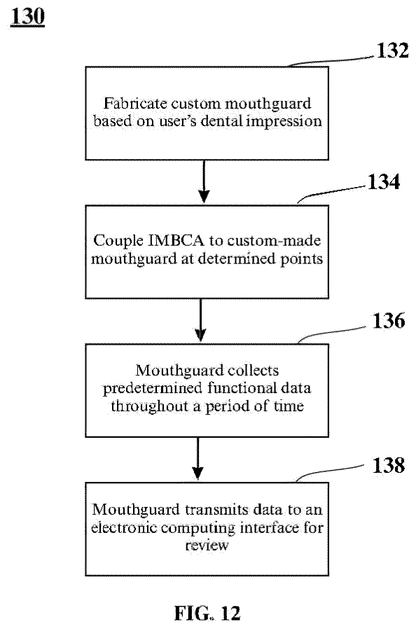

FIG. 13 is a flow diagram showing a first illustrated embodiment of a process 140 for using the intraoral multisensory device and system 10 in accordance with an implementation of this disclosure. The instrumented mouth guard device 12 is charged and synced with the system's 10 software application 18 to update the device's 12 settings when an update is available 142. The user puts on the instrumented mouth guard device 12 144 and the device 12 collects data from the user while being worn 146. The instrumented mouth guard device 12 then transmits the data collected and synchs with the system's 10 software application 18 148 in real-time and/or after the user is done wearing the device 12, such as after sleeping or participating in an athletic event.

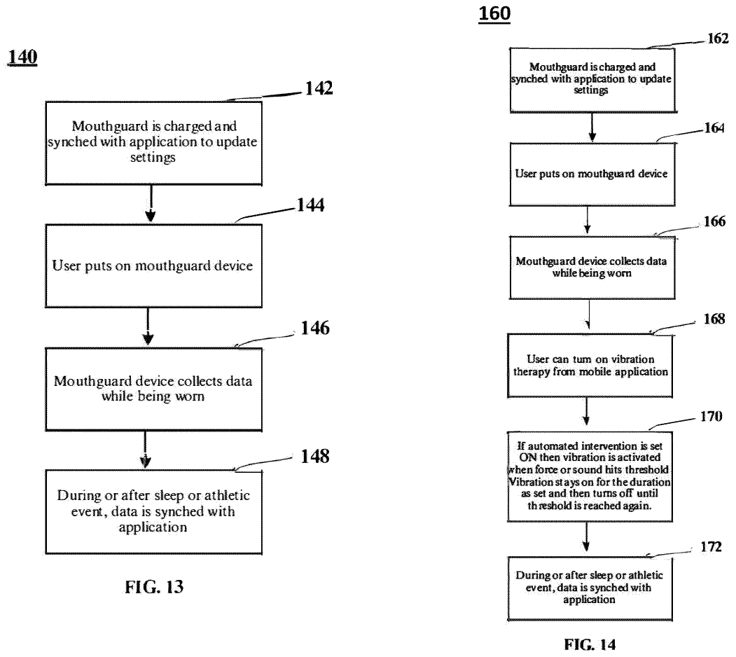

FIG. 14 is a flow diagram showing a second illustrated embodiment of a process 160 for using the intraoral multisensory device and system 10 in accordance with an implementation of this disclosure. The instrumented mouth guard device 12 is charged and synced with the system's 10 software application 18 to update the device's 12 settings when an update is available 162. The user puts on the instrumented mouth guard device 12 164 and the device 12 collects data from the user while being worn 166. The user may turn on the instrumented mouth guard device's 12 vibration therapy using the mobile application 18 (FIGS. 11A-11D) associated with the user's device 168. If automated intervention is set to ON, vibration therapy is activated in the instrumented mouth guard device 12 when the device 12 detects a force and/or sound that reaches and/or exceeds the configurable predetermined force threshold and/or the configurable predetermined sound threshold 170. The instrumented mouth guard device 12 then transmits the data collected and synchs with the system's 10 software application 18 172 in real-time and/or after the user is done wearing the device 12, such as after sleeping or participating in an athletic event.

For simplicity of explanation, processes 130, 140, and 160 are depicted and described as a series of steps. However, steps in accordance with this disclosure can occur in various orders and/or concurrently. Additionally, steps in accordance with this disclosure may occur with other steps not presented and described herein. Furthermore, not all illustrated steps may be required to implement a method in accordance with the disclosed subject matter.

As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, “X includes at least one of A and B” is intended to mean any of the natural inclusive permutations. That is, if X includes A; X includes B; or X includes both A and B, then “X includes at least one of A and B” is satisfied under any of the foregoing instances. The articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment, aspect or implementation unless described as such.

While the present disclosure has been described in connection with certain embodiments and measurements, it is to be understood that the invention is not to be limited to the disclosed embodiments and measurements but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for collecting, processing, storing, and transmitting data associated with a user comprising:
fabricating an instrumented mouth guard device based on at least one of intraoral dimensions of the user and a specific need of the user;
identifying at least one component engagement site on the instrumented mouth guard device based on the specific need of the user;
mounting at least one intraoral multisensor circuitry system (IMCS) to the at least one component engagement site of the instrumented mouth guard device based on health monitoring needs of the user; and
monitoring the data received from the instrumented mouth guard device for an event involving the user, the instrumented mouth guard device adapted to initiate at least one mechanical feedback indicator on a remote receiving device in response to the event.

2. The method of claim 1, further comprising:
setting at least one threshold parameter based on biometric data associated with the user; and
comparing the data received from the instrumented mouth guard device to the at least one threshold parameter; the instrumented mouth guard device adapted to initiate the at least one mechanical feedback indicator on a condition that the at least one thresholder parameter is at least one of reached and exceeded.

3. The method of claim 1, the data comprising at least one of:
a pH level of the user's saliva based on data from salivary testing performed by the instrumented mouth guard device;
a sound intensity measured by the instrumented mouth guard device on a condition that the user is at least one of snoring and grinding their teeth;
at least one of a grinding force and a clenching force measured by the instrumented mouth guard device of the user's bite plate; and
at least one of a linear force, a rotational force, and a spatial force measured by the instrumented mouth guard device on a condition that the user experienced a high force impact event.

4. The method of claim 1, further comprising:
sending a notification to at least one of a local user and a remotely located user on a condition that the data exceeds a configurable predetermined threshold value.

5. The method of claim 1, further comprising:
sending a notification using a mechanical feedback indicator.

6. The method of claim 5, the mechanical feedback indicator comprising a vibration module.

7. The method of claim 1, further comprising:
sending a notification to at least one of a local user and a remotely located user using the remote receiving device.

8. A non-invasive intraoral multisensor system for collecting, processing, storing, and transmitting biological data associated with a user comprising:
an instrumented mouth guard device adapted to at least one of receive, process, store, and transmit the biological data, monitor at least one physiological parameter of the user; and
a remote receiving device in communication with the instrumented mouth guard device, the remote receiving device adapted to at least one of receive, process, and store the biological data received from the instrumented mouth guard device, and initiate therapeutic intervention using the instrumented mouth guard device.

9. The non-invasive intraoral multisensor system of claim 8, the instrumented mouth guard device comprising:
a main aligner body comprising a flexible material and including an anterior portion;
at least one of opposing evenly spaced arcuate peripheral sidewalls and U-shaped peripheral sidewalls, the sidewalls including a channel corresponding to the contour and definition of at least one of an upper jaw region and a lower jaw region of the user's dental arch;
a depressed region along a perimeter of the main aligner body, the depressed region disposed within the channel, the depressed region adapted to engage with and receive the user's teeth and to aid in maintaining the instrumented mouth guard device in a fixed position within the user's oral cavity during operational use;
at least one intraoral multisensor circuit system (IMCS) disposed within the instrumented mouth guard device, the IMCS comprising at least one electronic component, the IMCS adapted to detect, collect, and manage at least one health metric of the user;
a control unit comprising a taptic engine, a power source, and a device proximity sensor operably connected to the at least one IMCS, the taptic engine symmetrically mounted within the instrumented mouth guard device, and
the remote receiving device adapted to receive, process, and store intraoral sensor data received from the instrumented mouth guard device.

10. The non-invasive intraoral multisensor system of claim 8, the instrumented mouth guard device comprising at least one of a smart phone, tablet, and an electronic device capable of receiving and/or transmitting the data.

11. The non-invasive intraoral multisensor system of claim 8, further comprising:
a mobile application installed on the remote receiving device, the mobile application in communication with the instrumented mouth guard device, the mobile application adapted to provide a user interface.

12. The non-invasive intraoral multisensor system of claim 9, further comprising:
at least one intraoral sensor disposed on the instrumented mouth guard device, the at least one intraoral sensor adapted to generate user activated signals corresponding to a predetermined threshold event;
a microprocessor unit mounted to the at least one of intraoral sensor, the microprocessor unit adapted to receive signals from the at least one intraoral sensor, convert said signals into digital signal data, and communicate said digital signal data to the remote receiving device via at least one of a wired communication channel and a wireless communication channel; and
a mechanical feedback indicator disposed on the instrumented mouth guard device, the mechanical feedback indicator adapted to be activated in response to detection of a configurable predetermined event.

13. The non-invasive intraoral multisensor system of claim 12, the microprocessor unit comprising:
a microcontroller including an embedded analog-to-digital convertor adapted to monitor at least one signal received from the at least one intraoral sensor and convert the at least one signal into the digital sensor data;
a microprocessor adapted to transmit the digital sensor data to the remote receiving device; and
at least one of a local memory unit and a remote storage device, the remote receiving device adapted to at least one of store the digital sensor data to at least one of the local memory unit and the remote storage device and to process the digital sensor data and activate the mechanical feedback indicator to alert the user of the occurrence of the configurable predetermined event, at least one of the local memory unit and the remote storage device adapted to store instructions and intraoral sensor data awaiting transmission to the remote receiving device.

14. The non-invasive intraoral multisensor system of claim 8, the therapeutic intervention activated by the remote receiving device comprising the initiation of the mechanical feedback indicator.

15. The non-invasive intraoral multisensor system of claim 12, the therapeutic intervention provided by the mechanical feedback indicator comprising a vibration module implemented by the taptic engine based on user-configurable control settings.

16. The non-invasive intraoral multisensor system of claim 15, the vibration module comprising at least one user-configurable taptic mode setting including at least one of operational on/off mode and a plurality of vibration modes including vibration patterns, vibration intensity, vibration duration, and automated therapeutic intervention modes.

17. The non-invasive intraoral multisensor system of claim 8, the remote receiving device adapted to process the intraoral sensor data received from the instrumented mouth guard device and at least one of monitor, analyze, and diagnose a health status of the user based on the intraoral sensor data.

18. The non-invasive intraoral multisensor system of claim 12, the remote receiving device further adapted to provide an indication of a condition of the health status of the user through a user interface on the remote receiving device.

19. The non-invasive intraoral multisensor system of claim 13, the remote receiving device further adapted to receive at least one of pressure sensor data, force sensor data, pH sensor data, salivary testing data, audio sensor data, proximity sensor data, and inertial sensor data from the IMCS of the instrumented mouth guard device.

20. The non-invasive intraoral multisensor system of claim 8, the remote receiving device further adapted to initiate a therapeutic intervention on a condition that the health status of the user comprises an event.
